(19) United States
(12) Patent Application Publication
(10) Pub. No.: US 2006/0018788 A1
(43) Pub. Date: Jan. 26, 2006

(54) Method and apparatus for vapor, gas or gas-liquid treatment of surfaces and articles

(76) Inventors: Gustavo Jose Monico, Laguna Hills, CA (US); Christopher Reece Purvis, Irvine, CA (US)

Correspondence Address: ROBERT D. FISH;RUTAN & TUCKER LLP, 611 ANTON BLVD 14TH FLOOR, COSTA MESA, CA, 92626-1931, US

(21) Appl. No.: 11/187,793

(22) Filed: Jul. 22, 2005

Related U.S. Application Data
(60) Provisional application No. 60/590,796, filed on Jul. 23, 2004.

(51) Int. Cl. A61L 2/07

(52) U.S. Cl. 422/26; 422/1; 422/307

(57) ABSTRACT

The present invention provides a systems and methods in which H₂O₂ is decomposed using a catalyst to produce steam, and the steam is used to sterilize medical devices or other objects. The apparatus is preferably hand held, and has a steam port at one end. Objects to be sterilized are preferably contained in a pouch having a coupling adapted to couple to the steam port.

This application claims priority to provisional application Ser. No. 60/590,796 filed Jul. 23, 2004.

### FIELD OF THE INVENTION

The field of the invention is heat sterilization.

### BACKGROUND

There is an ongoing need for “ad hoc”, on-site sterilization that can be accomplished virtually anytime, anywhere. The need arises from several sources, including increased demand for medical care, increased liability, and migration of care from expensive centralized facilities to less expensive outpatient clinics. Medical device manufacturers drive these demands further as they seek to switch to “re-usable” rather than “disposable” instruments to alleviate waste issues.

Even within a given facility, the need for on-site sterilization capability has increased in recent years as a result of pressures to: 1) increase service throughput, 2) accommodate odd-shaped items and batch sizes, and 3) comply with strict regulations. At the same time these same entities must reduce 4) cost, 5) turn-around times, 6) inventory on hand, 7) error rates, and 8) use of space. This impels the need for a low-footprint, low-cost means of sterilization that provides on-demand, steam-based, variable-sized batch sterilization.

Additionally some entities such as field military units have urgent needs for low-cost, autonomous, and portable sterilization units. In this same vein, Third World countries where access to electricity and safe water is uncommon (e.g. much of Africa; parts of Asia, Latin America) also seek an inexpensive, autonomous, and versatile solution.

These demands have been addressed in numerous different ways over the years. Among the more important methods are: heat-based (steam, dry heat, chemical vapor, microwave), low temperature methods (low-pressure and temperature vapor), low temperature gas methods (Ethylene oxide aka EtO), radiation methods (e.g., electron beam, gamma rays). Each of these methods has their strengths and drawbacks.

Among the listed methods' strengths: heat-based methods are tried-and-true and reliably sterilize any articles that can tolerate the sterilization environment in a short time period (e.g. 20-30 min). This is basic sterilization at its best. Low temperature methods are more gentle and usually allow sterilization of articles made from a wider range of materials—an example is vapor-plasma (e.g. H₂O₂ vapor) which can sterilize many articles reliably, also within 30 min. Gas methods such as ethylene oxide kill a wide range of pathogens, are gentle to most materials, and sterilize within 30 min. Radiation is inexpensive when used for high-volume operations, and is very fast and reliable.

Among the listed methods' drawbacks: not all materials can withstand the temperatures (and moisture) of a tried and true heat-based method such as steam. Older low-temperature methods such as soaking in glutaraldehyde require many hours or even days to complete the sterilization process. Newer low-temperature methods (vapor-H₂O₂) require vacuum, elaborate bulky machinery, have trouble with deep lumens in instruments, and require expensive equipment. Gasses such as EtO are toxic and carcinogenic, highly flammable and explosive, can leave toxic residue on articles (this require additional aeration cycles, therefore much more time to finish the operation), are expensive to handle and dispose of properly, and their use is sometimes banned entirely by communities. Radiation and electron beam methods also pose risks, can damage materials (e.g. certain plastics, even foods), require very bulky infrastructure, and require very high capital outlays.

Hydrogen Peroxide alone. Hydrogen peroxide (H₂O₂) has been known to have bactericidal properties and has been used in solutions to kill bacteria on various surfaces. U.S. Pat. No. 4,437,567 discloses the use of aqueous hydrogen peroxide solutions at low concentrations, i.e., 0.01% to 0.10% by weight, to sterilize packaged products for medical or surgical use. At room temperature sterilization requires at least 15 days. At higher temperatures sterilization can be accomplished in approximately one day. Additionally straight H₂O₂ can leave residuals that must be removed from food or applied in special ways to sensitive components such as contact lenses. See U.S. Pat. Nos. 4,368,081 and 5,468,448. A Johnson & Johnson™ product marketed under the trade name Sterrad™ uses substantially ambient temperature H₂O₂ as a chemical sterilizing agent. Unfortunately, such “low-temperature” sterilization has numerous limitations, including for example a difficulty in sterilizing instruments with deep lumens, incompatibility with common materials (e.g. paper), and excessive bulk and expense.

Hydrogen Peroxide Vapor. U.S. Pat. Nos. 4,169,123; 4,169,124 and 4,230,663 disclose the use of hydrogen peroxide in the gas phase at temperatures below 80.degree. C. and concentrations of 0.10 to 75 mg H.sub.2 O.sub.2 vapor/L for sterilization and disinfection. Depending upon concentration and temperature, sterilization times are reported to vary from 30 minutes to four hours.

Plasma alone. The use of plasma to sterilize containers was suggested in U.S. Pat. No. 3,383,163. Plasma is an ionized body of gas which may be generated by the application of power from different sources. The ionized gas will contact microorganisms on the surfaces of the items to be sterilized and effectively destroy the microorganisms.

Plasma Generation. U.S. Pat. No. 3,851,436 discloses the use of radio frequency generators to produce such plasmas from inert gases such as argon, helium or xenon. U.S. Pat. No. 3,948,601 also discloses the use of a radio frequency generated plasma which ionizes argon, nitrogen, oxygen, helium or xenon. The processes set forth in the above-mentioned patent require the direct contact of the plasma on the surface of the product to be sterilized, which product is not packaged at the time of sterilization. The commercial sterilization procedures used to sterilize disposable medical goods generally require that the medical goods be packaged prior to sterilization because of the possibility of contamination by microorganisms if the products are packaged subsequent to sterilization.

Plasma Glutaraldehyde. U.S. Pat. No. 4,207,286 discloses a gas plasma sterilization system which uses glutaraldehyde as the gas used in a plasma sterilization system. The item to be sterilized is placed in an unsealed container or package and then subjected to the sterilization cycle. When the sterilization cycle is completed, the containers are sealed. The container must be opened during the sterilization cycle to allow the gas to flow into the interior of the package or container to allow contact of the gas with any microorganisms which may be on the surface of the item to be sterilized.

Plasma Oxygen. U.S. Pat. No. 4,321,232 discloses a plasma sterilization system in which the item to be sterilized is placed in a package made from a porous material. The gas used in the process is oxygen, and it is indicated that sterilization can be accomplished through the porous packaging within 60 minutes.

Plasma Sterilization. U.S. Pat. No. 4,348,357 discloses a plasma sterilization procedure using oxygen, nitrogen, helium, argon or Freon™ as the gas. The pressure is pulsed, that is, the pressure within the container is alternately increased or decreased in a cyclic fashion. In addition, the plasma may be de-energized during the pressure fall portion of the pressure cycle to reduce the heating effect on the article to be sterilized.

Japanese Application Disclosure No. 103460-1983 discloses a plasma sterilization process in which the gas consists of nitrous oxide or a mixture of nitrous oxide with another gas such as oxygen, helium or argon. Japanese Application Disclosure No. 162276-1983 discloses the sterilization of foods using nitrogen oxide gas or mixtures of nitrogen oxide gas and ozone in a plasma. All of these prior plasma sterilization systems have not been put into wide commercial use because of the limitations on the time required to effect sterilization, the temperature obtained in the sterilization process or the particular requirements of some of the processes that would require post-sterilization packaging.

Hydrogen Peroxide Vapor and plasma. U.S. Pat. No. 4,756,882 combines hydrogen peroxide and plasma. A search on this patent found 91 successor patents that refer to this patent. Note as of this writing there is a popular new product line from Advanced Sterilization Products, a subsidiary of Johnson & Johnson, that features this sterilization approach.

Hydrogen Peroxide and Ultraviolet. The use of ultraviolet radiation with hydrogen peroxide for improved antimicrobial activity has been disclosed in U.S. Pat. Nos. 4,366,125 and 4,289,728. The lack of penetration by UV radiation below the surface of the object to be sterilized limits the application of this effect to clear solutions or surfaces that can be directly exposed to the radiation. Articles in an opaque package, or articles in a clear package that absorbs UV light could not be sterilized

Microwave Sterilization. U.S. Pat. No. 3,753,651 of Boucher shows in general a household oven type of microwave source for providing energy that irradiates the instruments to kill the bacteria and other micro-organisms. An obvious disadvantage of Boucher is that if the instruments are metallic, as they often are, partial discharge at sharp points will be detrimental and destructive to the instruments.

Microwave or Infra-red Sterilization. U.S. Pat. No. 4,400,357 of Hohmann contemplates in one embodiment, an IR source for heating the reactant liquid to vaporize it for sterilization of medical instruments. Another embodiment suggests that a microwave source and cavity resonator might be employed in place of an IR source. The patent, however, fails to disclose an efficient coupling between the microwave source and the reactant liquid whereby the latter may be quickly and efficiently vaporized. Without this efficient coupling, the microwave sterilizer of Hohmann would be subject to the time consuming period for vaporization that is found in sterilizers currently in use employing other types of heating elements or result in damage to or destruction of the microwave source.

Flexible Enclosures. U.S. Pat. No. 5,871,702 of Kutner, et. al., proposes a flexible pouch within a rigid enclosure, namely, a microwave-oven like device that heats liquid within the pouch to produce sterilizing treatment media within the flexible enclosure. U.S. Pat. No. 5,422,130 of Fox, et. al., proposes a semi-continuous method and apparatus for sterilizing food in flexible packages and bringing said packages to cool state without collapsing the package.

Thus, there is a continuing need for new apparatus and methods for sterilization.

### SUMMARY OF THE INVENTION

The present invention provides systems and methods in which hydrogen peroxide or other compound is used as a “fuel” which decomposes in the presence of a catalyst in a manner that produces heat. In the case of H₂O₂, the compound advantageously decomposes into steam (vapor) and oxygen, which in conjunction with the heat provides steam at sufficient temperature to produce the desired sterilization.

Preferred embodiments are compact, and need no electricity or water; the exothermic decomposition providing all the vapor and heat required to operate the device. Preferred embodiments can optionally utilize detachable flexible enclosures as the vessel in which sterilization takes place. Exemplary enclosures include a bag or wide, flexible hose. These flexible enclosures can contain any suitable number of articles, and advantageously can include instruments that vary widely in size, shape, and number. In some instances the enclosures can include smaller gas- or steam-permeable bags. Once sterilization is complete these bagged articles can be removed and the outer flexible enclosure re-used if appropriate.

It is also contemplated that embodiments of the inventive subject matter can include simple temperature and pressure sensors and feedback control. In preferred embodiments we generate a treatment media of steam by bringing H₂O₂ into contact with a catalyst, or vice-versa; moreover, said use of H₂O₂ means the apparatus can produce abundant hot steam using no external water or power. Although H₂O₂ and steam are preferred, in principle any liquid that decomposes exothermically into a gas in the presence of a catalyst can use the disclosed invention to treat articles or surfaces.

Various objects, features, aspects and advantages of the present invention will become more apparent from the following detailed description of preferred embodiments of the invention, along with the accompanying drawings in which like numerals represent like components.

### BRIEF DESCRIPTION OF THE DRAWING

FIG. 1 is a schematic of a device that is treating a surface in an ambient environment, with a fixed catalyst.

FIG. 2 is a schematic of a device that is treating articles in an enclosure, with a fixed catalyst.

FIG. 3 is a schematic of a device that is treating articles in a arbitrary enclosure, with a movable catalyst.

FIG. 4 is a schematic of a flexible enclosure with a sealable orifice.

FIG. 5 is a vertical cross-section of a device that is treating articles in an enclosure, with a fixed catalyst.

### DETAILED DESCRIPTION

In general, contemplated devices generate a treatment media (likely, a gas mixture) through exothermic decomposition of a liquid in the presence of a suitable catalyst. In our preferred embodiment we create a steam treatment media through use of H₂O₂ as a “fuel” that runs through a catalyst and decomposes into oxygen-rich steam; this steam in turn treats the object(s) in question. The associated chemistry is:
Hydrogen Peroxide+catalyst→Treatment Media(Steam+Nascent Oxygen)2H₂O₂+catalyst→2H₂O+2[O]+heat

Notably, our preferred embodiments do not use the liquid (preferably here, H₂O₂) directly as the treatment media—although it is possible to inject some un-decomposed H₂O₂ into the output flow of steam if said injection enhances sterilization treatment—and we do not heat water through use of common means to generate steam, i.e., we do not heat water to its boiling point via heating devices such as direct heating elements, infrared sources, or even microwaves.

Preferred embodiments in our invention return to the use of steam, but in a very compact form that requires no external water or power. In particular, use of hydrogen peroxide (H₂O₂) eliminates any environmental concerns either with the H₂O₂ as an input or with the water vapor and oxygen that form the treatment media and are process outputs.

The steam generator portion of the invention can be used at least three ways: 1) as a portable device used directly to treat non-enclosed articles (e.g. surfaces in the external environment); 2) as a portable device that connects directly to the disclosed invention's proposed flexible enclosures (which contain articles to be sterilized), or 3) as part of a stationary device, namely, as a component inside a traditional rigid autoclave, in order to provide the benefit of autonomy (no water or power required) with a traditional rigid enclosure.

We want to be clear about our use of decomposable liquids such as hydrogen peroxide (H₂O₂): in our preferred embodiments we DO NOT use these liquids (e.g., H₂O₂) directly as the treatment media; rather, we create a treatment media such as steam through use of the liquid as a “fuel” that runs through a catalyst and decomposes into treatment media (e.g., oxygen-rich steam in the case of H₂O₂) that in turn treats the object(s) in question.

Those skilled in that art will appreciate that it is possible to inject replacement (un-decomposed) H₂O₂ into the invention's steam output if evidence shows this produces faster or better treatment of sterilization of the articles or surfaces in question.

We also want to be clear that our method further differs from conventional means to generate steam, in which water is heated to its boiling point by conventional heating devices such as direct heating elements, infrared sources, or even microwaves (see below). Preferred embodiments of the present invention use controlled, exothermic decomposition of a liquid in the presence of a catalyst.

Since the goal is to generate steam by way of decomposition reaction, those skilled in the art will appreciate that substances other than H₂O₂ can be used as the “fuel” (technically, H₂O₂ is an oxidizer). These substances contain hydrogen and oxygen—e.g., organic compounds such as methanol—and in the presence of oxygen and a catalyst can be decomposed into steam and other by-products, preferably by-products that are non-toxic and non-corrosive.

A more complete appreciation of aspects of the present invention, and many of the attendant advantages thereof, will be readily understood by reference to the following detailed description when considered in connection with the accompanying drawings in which:

In FIG. 1 a sterilization device 100 is being used to sterilize a surface of an object 190 in an ambient environment. The sterilization device 100 generally includes reaction chamber 110, a catalyst or other reducing agent 120, fuel compartment 130, and port 180.

Use of some fuels (i.e. oxidizers) will require secondary and/or external reactants such as O₂ or air. Therefore, it is contemplated that the sterilization device can optionally have additional reactant compartment 140, which stores secondary reactant needed for decomposition reaction. It is also contemplated that an external reactant input 150 can be coupled to the device, thereby allowing introduction of oxygen or air into the reaction chamber 110.

In the particular example of FIG. 1, H₂O₂ from compartment 130 is brought into contact with the catalyst 120 in a controlled manner. The catalyst position is fixed relative to the reference frame of the sterilization device.

The most suitable “fuel” is H₂O₂ which can be decomposed by a number of catalysts, including silver, MnO₂, Ruthenium, Platinum, Palladium, Gold, Rhodium, or combinations of these; silver-plated nickel screen; and proprietary catalysts such as Shell 405 (an iridium-based catalyst from Shell™ Oil Company) and General Kinetics Type 1 (from General Kinetics™ Inc., 22661 Lambert St, Lake Forest, Calif. 92630). Those skilled in the art will appreciate that not all catalysts will work well with all fuels (or oxidizers). For example, where the substance is H₂O₂, the most suitable catalysts are General Kinetics Type 1, Shell 405, MnO2, and Silver; less suitable catalysts are Ruthenium, Platinum, Palladium, Gold, and Rhodium. To our knowledge the General Kinetics Type 1 catalyst is most superior for use in decomposing H₂O₂. H₂O₂ can be present in any suitable volumes and concentrations. In preferred embodiments, H₂O₂ volumes ranged from about 10 ml to about 500 ml. These and all other ranges set forth herein are inclusive of their endpoints unless the context indicates otherwise. Preferred concentrations of H₂O₂ range from about 55% to about 90%, more preferably between about 65% and about 75%, and most preferably between about 69% and about 72%.

While catalyst 120 is shown as disposed within reaction chamber 110, those skilled in the art will immediately appreciate that catalyst 120 can be stored in a separate compartment and then introduced into the reaction chamber when needed. Similarly, while the figure shows compartments 130 and 140 as located externally to reaction chamber 110, those skilled in the art will appreciate the possibility that these compartments can be disposed within the reaction chamber 110.

In preferred embodiments, the device 100 has feedback control 160 coupled to relevant input points 130, 140, 150, which sense temperature and/or pressure of the output (steam) and adjust the net rate of release of the fuel to the catalyst. Those skilled in the art should appreciate that all types of feedback mechanisms can be used to monitor and control the steam output, including feedback mechanisms that provide for fully automated control.

Operation of the sterilization device is straightforward. The user of the device introduces H₂O₂ stored in compartment 120 into reaction chamber 110. H₂O₂ reacts with catalyst 120 and steam is produced as a result of the reaction. Steam exits port 180 and sterilizes object 190. Feedback control 160 senses the temperature and/or pressure of the steam. Base on the data collected by control 160, the device re-adjust the net rate of decomposition of H₂O₂ by changing the amount of H₂O₂ going into reaction chamber 110.

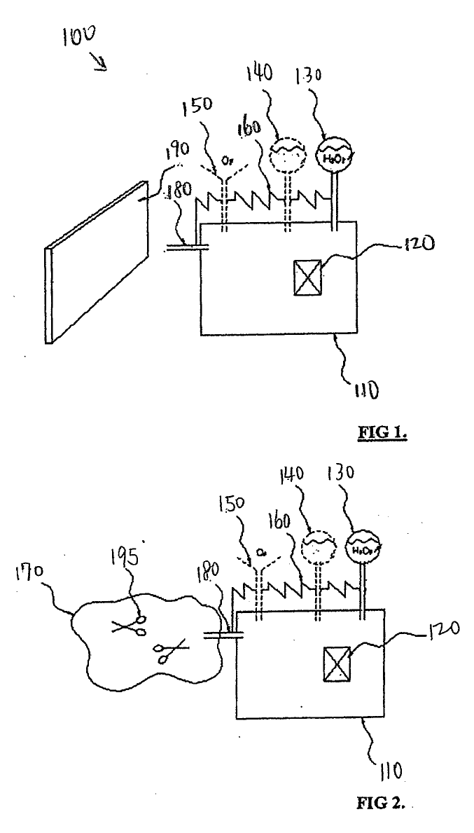

FIG. 2 shows an embodiment substantially similar to that shown in FIG. 1, but further including an enclosure 170 to sterilize a plurality of articles 195. The size and shape of the enclosure is arbitrary, and those skilled in the art will immediately appreciate that all practical sizes, shapes, and configurations are possible, and are limited only by the ability of the sterilization device to supply steam of appropriate temperature and pressure into the enclosure 170.

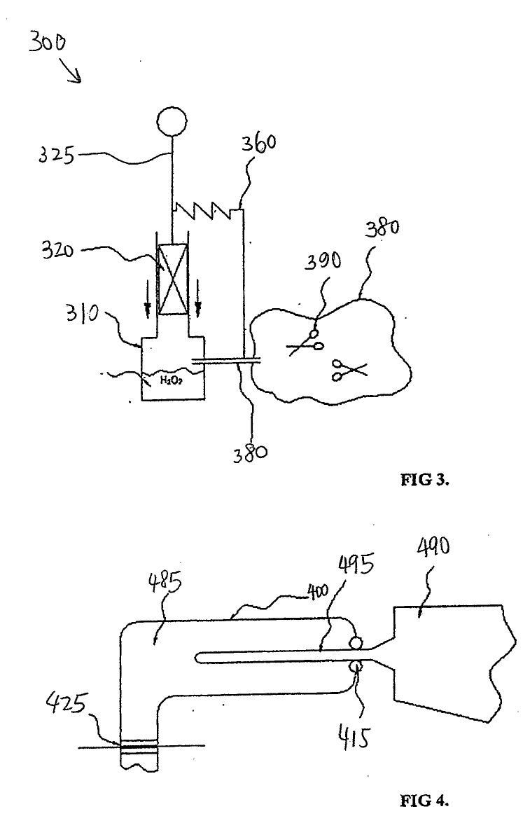

In FIG. 3 a device 300 has a reaction chamber 310 containing H₂O₂ that remains largely stationary, and the catalyst 320 is brought in contact with H₂O₂ in a controlled manner. Resulting steam is released from port 380 into enclosure 380 to sterilize objects 390. The device has feedback control 360 to sense temperature and pressure within an enclosure 380 and adjust the net rate of decomposition of liquid accordingly. The rate of decomposition can be adjusted by manually moving handle 325 which moves the catalyst 320 in and out of reaction chamber 310.

Although handle 325 suggests a manually operated system, those skilled in the art will appreciate that the handle can be fully automated, and can be operated, for example, by a central processing unit that electronically monitors and controls the rate of decomposition according to a pre-set value.

In FIG. 4 a flexible enclosure 400 has two openings, one for entry of steam and one for exit of steam. One opening, having connector 425, connects to a sterilization device and receives treatment media, (e.g., steam). Another opening, 415 is large enough to receive an object to be sterilized or otherwise treated. Both openings 425 and 415 are sealable following treatment. The treated (e.g. cleaned, sterilized) the article remains sealed within enclosure 400 ready for use.

The enclosure can comprise of any suitable material or materials, including, for example, metal, polymers, and so forth. In a preferred embodiment the enclosure is made of plastic. Further, the enclosure can have suitable size and configuration, including especially configurations that are sized and dimensioned to accommodate the shape of the portion 495 of an object 490 to be sterilized.

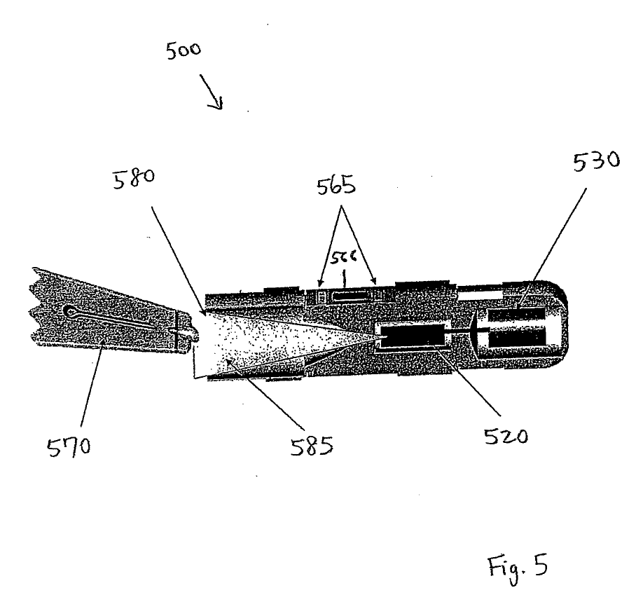

In FIG. 5 a contemplated sterilization device 500 has a H₂O₂ canister 530, a catalyst bed 520, a port 580, enclosure 570, and controllers 565. To sterilize an object placed in the enclosure 570, H₂O₂ is controllably released into catalyst bed 520 to react with silver catalyst. Decomposition reaction begins and resulting steam 585 is released through port 580 into enclosure 570.

Controllers 565 can control any useful parameter, including time, temperature, or pressure. Such controllers operate by monitoring appropriate sensors (not shown) and opening or closing a valve (not shown) that alters flow of fuel from canister 520 to reaction chamber 520. An optional gauge 566 can be analog, digital or some combination of the two.

Thus, specific embodiments and applications of sterilization have been disclosed. It should be apparent, however, to those skilled in the art that many more modifications besides those already described are possible without departing from the inventive concepts herein. The inventive subject matter, therefore, is not to be restricted except in the spirit of the appended claims. Moreover, in interpreting both the specification and the claims, all terms should be interpreted in the broadest possible manner consistent with the context. In particular, the terms “comprises” and “comprising” should be interpreted as referring to elements, components, or steps in a non-exclusive manner, indicating that the referenced elements, components, or steps may be present, or utilized, or combined with other elements, components, or steps that are not expressly referenced. Where the specification claims refers to at least one of something selected from the group consisting of A, B, C . . . and N, the text should be interpreted as requiring only one element from the group, not A plus N, or B plus N, etc.

What is claimed is:

1. A method for treating a surface of an object, comprising:
combining a liquid with a catalyst, the liquid and catalyst selected to react exothermically, and to produce an amount of heat; and
maintaining the surface in contact with the heat for a sufficient processing period to sterilize the surface.

2. The method of claim 1, further comprising, placing the object in a container, and introducing the liquid into the container.

3. The method of claim 2, further comprising the step of adding replacement liquid to into the container during the processing period.

4. The method of claim 2, wherein the container comprises a flexible pouch.

5. The method of claim 2, wherein the flexible pouch includes a permanently sealable orifice.

6. The method of claim 1, wherein the liquid comprises H₂O₂.

7. The method of claim 4, wherein the liquid comprises H₂O₂ at a concentration of at least 65%.

8. A device, comprising:
a source of an oxidizer operatively coupled to a reaction chamber that contains a catalyst that reacts with the fuel to produce steam;
a controller that controls a rate of production of the steam; and
a steam outlet port.

9. The device of claim 8, wherein the fuel comprises H₂O₂.

10. The device of claim 8, wherein the fuel comprises an organic compound.

11. The device of claim 8, wherein the catalyst comprises at least one of MnO2, Silver, Ruthenium, Rhodium, Platinum, Palladium, Gold, or proprietary catalysts such as General Kinetics Type 1™, Shell 405™.

12. The device of claim 8, wherein the fuel and the reaction chamber are contained within a housing sized and dimensioned to be carried by hand.

13. The device of claim 8, wherein the controller comprises a timer.

14. The device of claim 8, wherein the controller comprises a temperature controller.

15. The device of claim 8, wherein the controller comprises a pressure controller.

16. A system for sterilizing objects, comprising a device according to claim 8, and a sterilization container having a coupling removably attachable to the steam outlet port.

17. The system of claim 16, wherein the coupling is sealable against bacterial intrusion.

18. The system of claim 16, wherein the sterilization container comprises a flexible bag.
